(19) United States
(12) Patent Application Publication
(10) Pub. No.: US 2026/0069359 A1
(43) Pub. Date: Mar. 12, 2026

(54) ENDOVASCULAR AORTIC SEPTOTOMY DEVICE FOR TYPE B AORTIC DISSECTION

(71) Applicant: The Regents of The University of Michigan, Ann Arbor, MI (US)

(72) Inventors: Shinichi FUKUHARA, Ann Arbor, MI (US); Jeffrey Stephen PLOTT, Algonac, MI (US); Aaron KEHRER, Ypsilanti, MI (US); Nathaniel JOHNSON, Ypsilanti, MI (US)

(21) Appl. No.: 18/871,907

(22) PCT Filed: Jun. 9, 2023
(86) PCT No.: PCT/US2023/024934

§ 371 Date: Dec. 5, 2024

Related U.S. Application Data
(60) Provisional application No. 63/351,462, filed on Jun. 13, 2022.

(51) Int. Cl. A61B 18/22; A61B 18/00

(52) U.S. Cl. CPC A61B 18/22; A61B 2018/00601

(57) ABSTRACT

An aortic septotomy device for cutting aortic tissue between a true lumen and a false lumen. The aortic septotomy device having a handle device having a jaw actuator member and an optional cutter actuator member; a distal head assembly having a pair of jaw members movable between an opened position and a clamping position and a cutting mechanism movable within a channel; and a sheath member extending from the handle device to the distal head assembly; wherein at least one of the pair of jaw members is movable in response to actuation of the jaw actuator member to selectively grasp aortic tissue, and the cutting mechanism is movable within the channel in response to actuation of the cutter actuator member to cut at least a portion of the aortic tissue while the pair of jaw members grasps the aortic tissue.

### CROSS-REFERENCE TO RELATED APPLICATIONS

This application is a PCT International Application of U.S. Provisional Patent Application No. 63/351,462 filed on Jun. 13, 2022. The entire disclosure of the above application is incorporated herein by reference.

### FIELD

The present disclosure relates to aortic septal cutters and, more particularly, relates to an aortic septotomy device to create optimized landing zones during endovascular aortic repair for chronic type B aortic dissection.

### BACKGROUND AND SUMMARY

This section provides background information related to the present disclosure which is not necessarily prior art. This section also provides a general summary of the disclosure, and is not a comprehensive disclosure of its full scope or all of its features.

As illustrated in FIG. 1, aortic dissections or tears typically occur due to weakening of the inner layer of the artery wall. If left untreated, the tear can enlarge and blood can pass through the tear into the middle layer of the wall, causing the layers to separate from one another, or dissect. This can lead to the formation of a false lumen between the layers preventing proper blood flow to the body.

As is understood in the art, the separation of the inner layer of the aorta forms a flap, called a septum, having a plurality of holes that permit blood flow between the true lumen and the false lumen. Blood can pool in the false lumen. Over time, the blood in the false lumen can back up and clot, cutting off blood flow to organs and further weakening the aortic wall, or result in rupture of the aorta.

Aortic dissections that occur in the ascending part of the aorta are called type A; those in the descending aorta are type B. Type B aortic dissection originates in the descending aorta, which extends from the arch at the top of the ascending aorta—the part that extends upward from the heart—to the bottom section of the aorta, also known as the abdominal aorta. Chronic type B dissection can lead to an aneurysm-a bulge in the aortic wall—that can lead to an aortic rupture.

Persistent false lumen perfusion due to the presence of a thick aortic septum is a significant obstacle to successful thoracic endovascular aortic repair (TEVAR) for chronic type B aortic dissection (cTBAD). Although hospital survival has been good with open conversion after TEVAR, late survival was less than favorable, and many patients required additional operations. Many of the late open and endovascular operations were performed for progression of aneurysmal degeneration either related to the presence of a chronic dissection or a known connective tissue disorder consistent with the progressive nature of extensive aortic aneurysmal disease.

Endovascular surgeries are becoming more popular as a method for aorta repair. However, even with advancements in endovascular techniques, limitations remain. TEVAR is dependent on securing a stent-graft within the region of the aorta with the dissection. This can become difficult to perform for some patients with aortic dissection (AD) due to the thickness of the aortic dissection flap. In such cases, a procedure can be performed that cuts this flap so the blood will flow back into the inside (or true lumen) of the blood vessel. However, this procedure is risky and highly technically demanding. Current techniques for cutting the AD flap involve off-label usage of current endovascular devices which has made these procedures unproven, unreliable, and non-reproducible with the possible risk of lethal adverse effects such as full aortic rupture. There is currently an unmet need in the field of endovascular procedures for an endovascular device specifically designed to cut through the AD flap.

TEVAR for chronic type B aortic dissection remains controversial due to the presence of thickened aortic septum, which is considered a major cause of persistent false lumen perfusion despite TEVAR.

Devices for thoracic endovascular aortic repair for chronic type B aortic dissection have been introduced in the art. For example, there exists a laser aortic septotomy device that employs a laser system used for dissection. However, this laser-based septotomy device is technically demanding and requires the use of a complex laser system. Moreover, it has been found that there are significant limitations in connection with anatomical suitability. In fact, often the placement of the laser for dissection has resulted in malpositioning and ineffective treatment.

Accordingly, there exists a need in the art for an aortic septotomy device that is able to overcome the limitations of the prior art. Moreover, there exists a need in the relevant art to provide an aortic septotomy device that is configured to precisely and reliably cut aortic dissection to provide optimized landing zones during endovascular aortic repair for chronic type B aortic dissection.

According to the principles of the present teachings, an endovascular aortic septotomy device is provided for chronic type B aortic dissection configured to dissect the septum and optimize the landing zone for a wide spectrum of chronic aortic dissection pathologies. In some embodiments, the endovascular aortic septotomy device comprises a distal head assembly with a pair of jaw members actuated by a user between open and closed positions; a guidewire routed through each of the two jaw members; a cutting member positioned within and shielded by at least one of the jaw members during deployment; the cutting member is actuated back and forth through channels formed in the distal head assembly along the length of the distal head assembly. In some embodiments, bi-directional, reciprocating cutting action is provided for tough “chronic” septum (as opposed to an easier-to-cut septum in an “acute” dissection); a flexible tip portion on at least one jaw member to aid entry into the false lumen; and a cutting member shield (potentially at the jaw pivot), and the cutting member extending past the shield to ensure a complete cut of the tissue grasped between the jaw members.

In some embodiments, the endovascular aortic septotomy device is configured to provide the ability to grab onto the aortic dissection flap and cut through it. This device shields the cutting element to prevent damaging any tissue on the way to the aortic dissection flap. Once at the aortic dissection flap, the endovascular aortic septotomy device will slide along guidewires which help position the top and bottom jaw members on either side of the aortic dissection flap. The jaw members can then grab onto the aortic dissection flap. Once attached, the hidden cutting element will slide along a channel or slot within the jaws, thereby cutting the retained aortic dissection flap. The device may then be advanced forward so the process can be repeated. Once the cutting is complete, the endovascular aortic septotomy device is removed. The process of cutting the aortic dissection flap will decrease the risk of aortic rupture and create a better attachment site, landing zone, for the stent-graft.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 illustrates classifications of aortic dissection as understood in the art;

FIG. 2 illustrates a type B aortic dissection as understood in the art;

FIG. 3 is a schematic illustration of the aortic septotomy according to the principles of the present teachings;

FIG. 4 is a perspective illustration of an endovascular aortic septotomy device according to some embodiments of the present teachings;

FIG. 5 is a schematic illustration of a cutting step of the present teachings;

FIG. 6 is a series of illustrations showing the procedural insertion, placement, gripping, and cutting steps of the present teachings;

FIG. 7 is an illustration showing an optimized landing zone following completion of the methods of the present teachings;

FIG. 8A is a partial cross-sectional illustration of the handle and jaw and cutting member system employing a ratchet and pawl system in an opened position;

FIG. 8B is an enlarged illustration of the distal head assembly of FIG. 8A;

FIG. 9A is a partial cross-sectional illustration of the handle and jaw and cutting member system employing a ratchet and pawl system in a closed position;

FIG. 9B is an enlarged illustration of the distal head assembly of FIG. 9A;

FIG. 10A is a partial cross-sectional illustration of the handle and jaw and cutting member system employing a ratchet and pawl system in a closed and cutting position;

FIG. 10B is an enlarged illustration of the distal head assembly of FIG. 10A;

FIG. 11 is a perspective illustration of an endovascular aortic septotomy device according to some embodiments of the present teachings;

FIG. 12A is a perspective illustration of an endovascular aortic septotomy device having a laser according to some embodiments of the present teachings; and

FIG. 12B is a partial cross-sectional illustration of the handle and jaw and cutting member system having a laser in a closed position. Corresponding reference numerals indicate corresponding parts throughout the several views of the drawings.

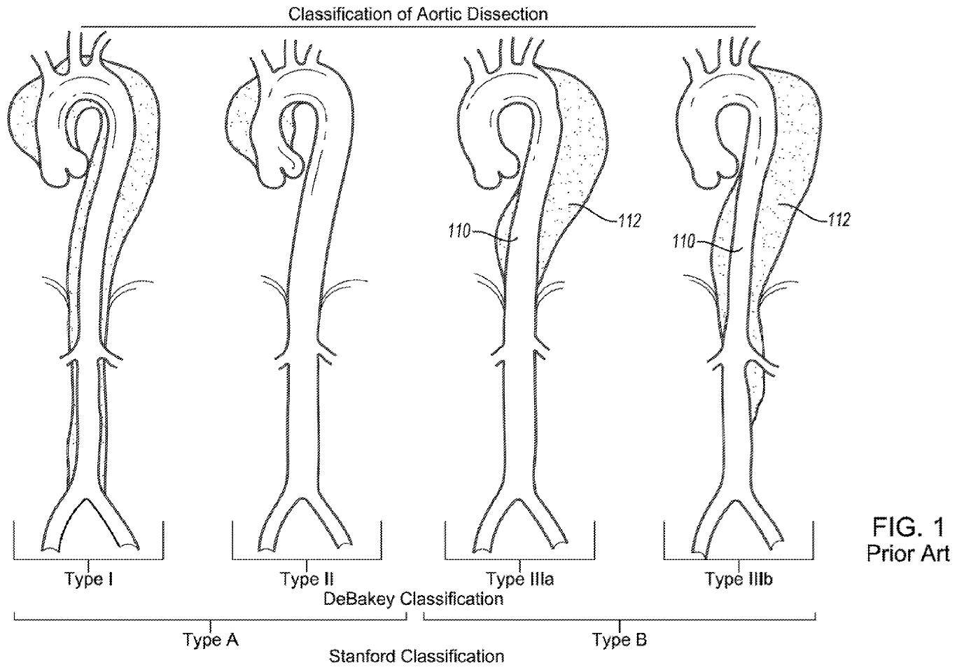

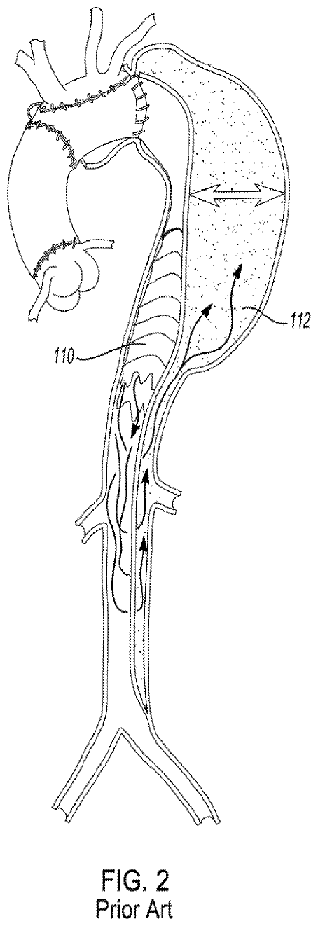

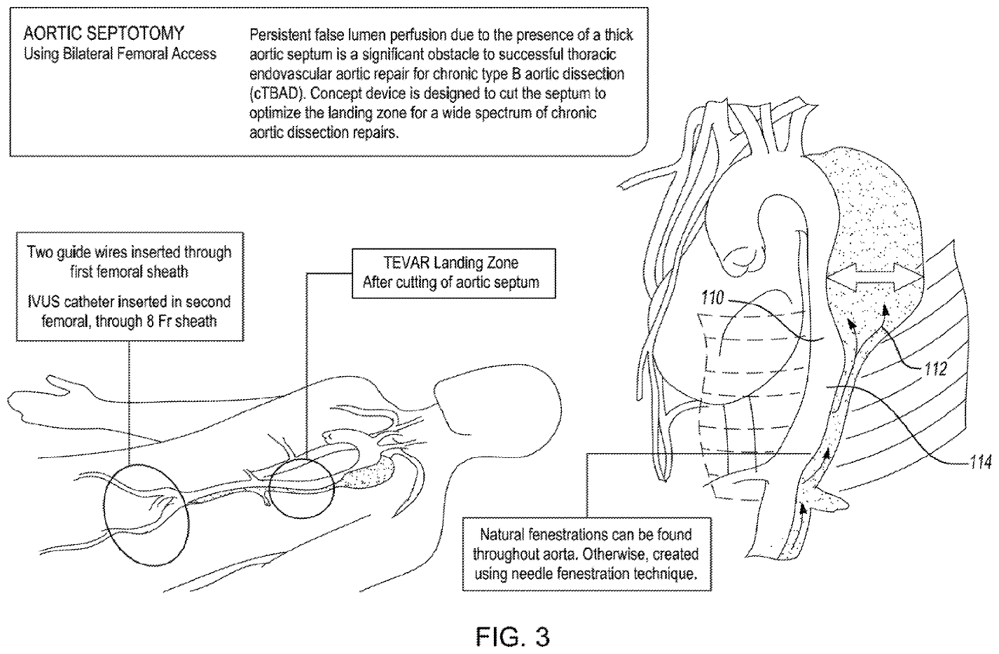

### DETAILED DESCRIPTION

Example embodiments will now be described more fully with reference to the accompanying drawings.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

### Technical Overview

As described herein, Aortic dissections (AD) are due to a tear in the innermost lining of the aorta allowing blood to flow into the lining of the blood vessel itself. This leads to separation of the innermost and middle layers of the aorta. Aortic dissections can lead to aortic aneurysms or aortic rupture and, moreover, can severely impair blood flow to vital organs and extremities.

While some aortic dissections can be managed with medication and lifestyle changes, other cases require medical intervention to repair the damaged aorta. These surgeries tend to involve the placement of an aortic graft that attaches above and below the dissection to prevent blood from flowing into the false lumen. These surgeries can fail due to the presence of the thick aortic dissection flap and persistent blood flow into the false lumen. For a significant number of patients, they are considered inoperable due to improper sites for attachment of the stent-graft above and below the dissection.

In both of these cases, a procedure can be done to enable successful aortic repair. The procedure involves cutting the lining that separates the true lumen and the false lumen (called aortic septum) to lower the pressure in the false lumen and provide better attachment sites for the stent-graft. Unfortunately, this is a high-risk procedure for the patient and limited advancement in new medical devices have kept this as a last resort procedure.

However, according to the principles of the present teachings, an endovascular aortic septotomy device 10 is provided that enables safe and reproducible aortic septotomy procedures. The endovascular aortic septotomy device 10 provides a new and novel endovascular aortic septal cutting device. In some embodiments, the endovascular aortic septotomy device 10 comprises a knife or cutting device that is moveably contained within a guard on the device to generally or completely prevent the cutting device from cutting tissue during the process of delivering it to the site of the dissection.

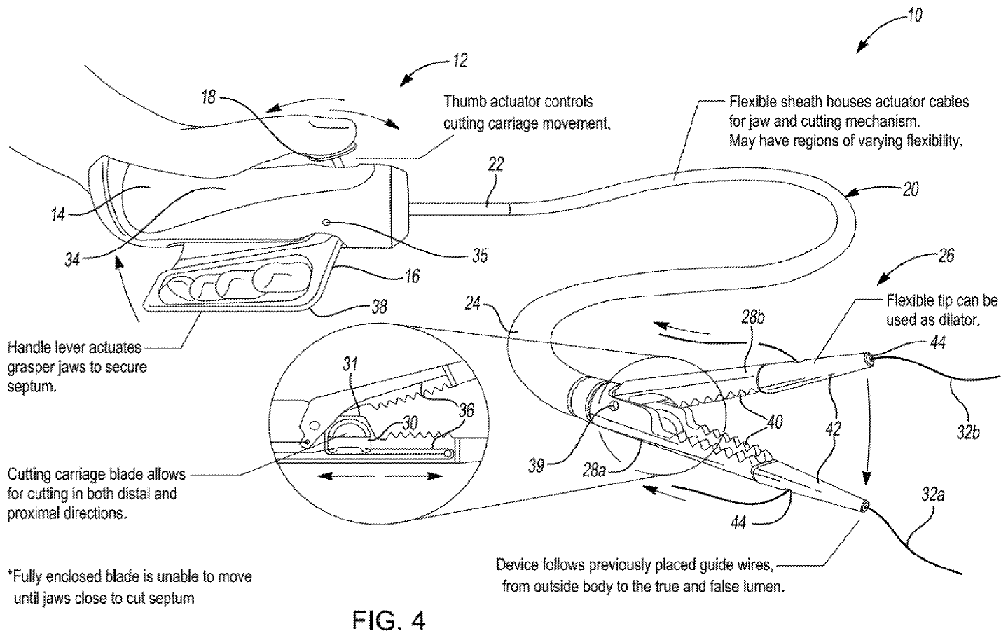

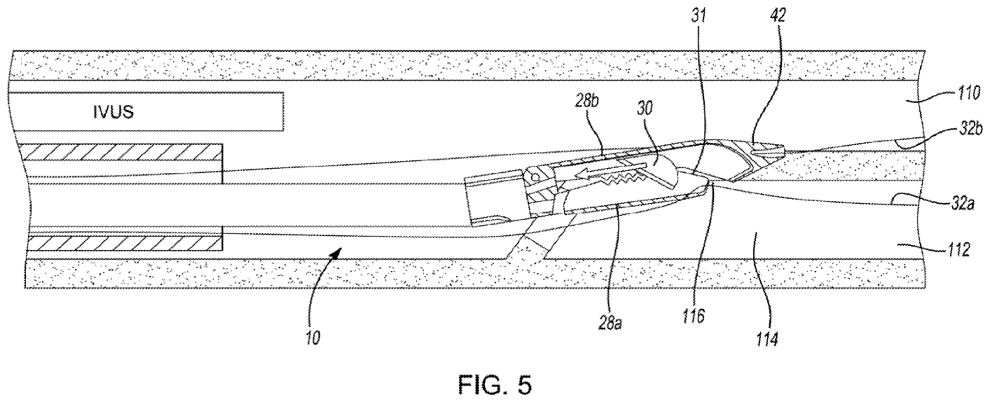

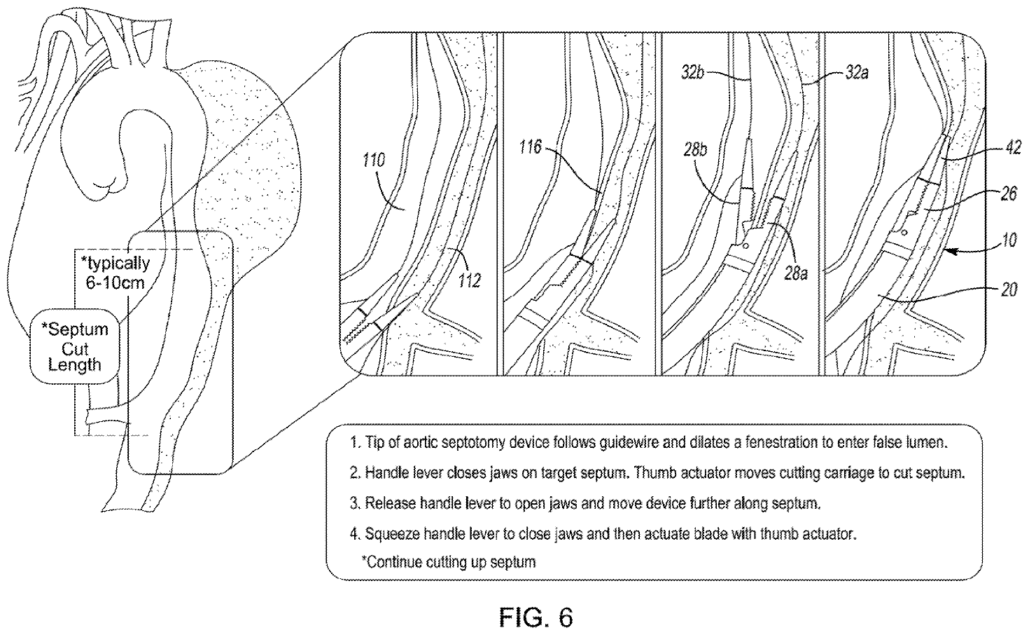

As will be discussed in greater detail herein, in some embodiments as illustrated in FIGS. 4-7, the endovascular aortic septotomy device 10 can comprise a handle device 12 having a grip portion 14, a jaw actuator member 16, and a cutter actuator member 18; a flexible sheath or catheter 20 extending from the handle device 12 on a proximal end 22 and terminate at a distal end 24; and a distal head assembly 26 extending from the distal end 24 of the flexible sheath 20. In some embodiments, distal head assembly 26 can comprise a pair of pivotally coupled jaw members 28a, 28b positionable between an opened or unclamping position (see FIGS. 4 and 6) and a closed or clamping position (see FIGS. 5 and 6) and a cutting member 30 slidably disposed within the distal head assembly 26. In some embodiments, the distal head assembly 26 comprises guidewires 32a, 32b extending within the true lumen 110 and the false lumen 112 to guide the distal head assembly 26 to the target area 114. The pair of jaw members 28a, 28b are configured to hold the dissection flap 116 during a cutting procedure.

Generally, as will be discussed in greater detail herein, the distal head assembly 26 can be inserted via a pushing motion of the handle device 12. The pair of jaw members 28a, 28b can remain in the opened position. The guidewires 32a, 32b, each extending through one of the pair of jaw members 28a, 28b, serve to guide the distal head assembly 26 to the target area (i.e., the dissection flap extending between the true lumen 110 and the false lumen 112). Once in the target area, the pair of jaw members 28a, 28b are actuated into the clamping position and the cutting member 30 is actuated to extend and cut the dissection flap. The pair of jaw members 28a, 28b can be actuated into the opened position and the distal head assembly 26 can be further inserted to an advance position to repeat the cutting process along the target area. The endovascular aortic septotomy device 10 is then removed and the region of the aorta that had the dissection can now equalize pressure in the true and false lumen and provide a more suitable landing zone for a stent-graft.

### Technical Background

The aorta is the largest artery in the human body and is critical for supplying blood to the head, arms, legs, and abdominal organs. The aorta starts at the heart and travels to the pelvis. The aortic blood vessel has three walls, or layers, that give the vessel strength and flexibility to withstand the pressure produced by blood flow-intima (the innermost wall), media (the middle layer), and adventitia (the outermost wall). The lumen is the open space of the vessel where blood flows through. The intima is in direct contact with the blood.

An aortic dissection is a tear in the innermost lining of the aorta (intima) that allows blood to flow into the rest of the aortic layers. This new passage for blood is called a ‘false lumen’. Blood flow into this false lumen can create several problems, including but not limited to a puncture in the outermost layer of the aorta leading to a full rupture or blood flow causing pressure on adjoining arteries resulting in impaired blood flow. These problems can impact blood flow to vital organs leading to organ damage.

As discussed previously, there are two types of aortic dissection: Type A, occurring in the ascending aorta (e.g., in the chest) and typically near the heart requiring immediate medical intervention to repair the dissection; and Type B, occurring in the descending aorta (e.g., in the abdomen). If the Type B dissection is stable, then medication is the recommended treatment. If the Type B dissection is rapidly progressing in size, the aorta ruptures, or vital organs become threatened, then surgical intervention is needed to repair the aorta. Roughly 25% of patients with type B aortic dissections will develop complications that require surgical intervention.

Generally, aortic dissections are very rare, but are life-threatening. Approximately 4 out of 100,000 people will be diagnosed with an aortic dissection. Left untreated, aortic dissections can lead to death within 24-48 hours. Without treatment the mortality rate is 1-3%/hour for the first 24 hours, 30% at 1 week, 80% at 2 weeks, and 90% at 1 year. Even with surgical intervention, roughly 10-30% of patients will die following treatment due to complications during and after surgery.

### Types of Surgical Intervention for Treating Aortic Dissections

There are two general surgery options: open surgery or endovascular surgery. Which surgery is performed is based on several variables like location of the dissection, severity of the dissection, the patient's age, and the patient's overall health. Open surgery options typically include open surgery fenestration, which involves removal of the aortic dissection and false lumen, or open aortic reconstruction, which involves removal of an aneurysm that has formed as a result of the dissection and replaced with an aortic graft. Endovascular surgery options typically include endovascular aortic repair, which involves placing a stent that is covered in the aortic graft into the region of the aorta that has the dissection. This is attached above and below the aortic dissection so blood can now flow through the newly reconstructed true lumen. There are several types of endovascular aortic repair, including endovascular aortic repair (EVAR), which are used to treat patients with a dissection occurring in a section of the aorta located in the abdomen, and TEVAR, which is used to treat patients with dissections occurring in a section of the aorta located in the chest. Branched and Fenestrated EVAR and TEVAR can be used when the aortic dissection is in an area where placing a standard EVAR or TEVAR could block arteries branching off of the aorta. These stents have reinforced openings (fenestrations) or branches that allow them to be attached to other arteries that lead to other organs.

These surgeries involve the placement of an aortic stent-graft that attaches above and below the dissection to prevent blood from flowing into the false lumen. This lowers the pressure in the false lumen which can prevent further dissection and possible impairment of blood flow in other arteries that branch off of the aorta. These surgeries can fail due to the presence of the thick aortic dissection flap and persistent blood flow into the false lumen. For some patients it is not possible to place the stent-graft in a position that completely closes off the dissection. In these cases, pressure will still build up in the false lumen as blood continues to flow through.

In these circumstances, fenestration of the aorta can help wherein the lining that separates the true and false lumen is cut. However, fenestration is currently a high-risk procedure. Current techniques involve using a blind blunt wire cutting technique to create a more robust attachment site for the stent-graft during TEVAR procedures. This technique is challenging because the tissue between the true and false lumen is thick. The pressure in the aorta can push back on the wire, impairing manipulation by the physician. This can lead to possible life-threatening complications like embolization of the aortic fragment or a serious injury to the aorta that could lead to aortic rupture.

That is, in greater detail, endovascular stent grafting for the treatment of thoracic aortic aneurysm following aortic dissection is often times challenging because the aortic dissection flap limits the availability of proximal or distal stent graft landing zones. Endografting during TEVAR with a dissected distal landing zone frequently does not work due to a persistent retrograde filling and pressurization of the false lumen. As a result, considerable number of patients sustain serious aortic adverse events as the natural course of untreated dissected aorta or require morbid open procedures after repeated endovascular treatment failures. A hybrid strategy represented by open surgical fenestration followed by TEVAR has been reported, although this technique still imposes invasiveness on patients and may not be suitable for those with high risk medical profile.

Endovascular longitudinal fenestration of the aortic dissection flap (septum), essentially using a blind blunt wire cutting technique, has been described in the past to create a robust distal aortic landing zone for TEVAR. However, this technique is not reliable or reproducible for cutting the thick aortic septum between the true and false lumens. More importantly, restive resistance force against the wire may cause errors in manipulation and distal embolization of an aortic fragment, or serious aortic injuries that can lead aortic rupture during the procedure. These potential hazards related to this blind procedure have been significant obstacles to the expansion of this endovascular aortic longitudinal fenestration concept.

There are a significant number of patients with untreated aorta who were deemed inoperable due to the lack of proper landing zone. There is a clear unmet need exists in our aortic surgical community. In order to achieve a safe and reproducible aortic longitudinal fenestration, a simple and reliable device is provided herein specially configured to overcome the limitations of the prior art.

### Detailed Construction

In some embodiments, with continued reference to FIGS. 4-7, the endovascular aortic septotomy device 10 comprise a handle device 12 having a grip portion 14, a jaw actuator member 16, and a cutter actuator member 18; a flexible sheath or catheter 20 extending from the handle device 12 on a proximal end 22 and terminate at a distal end 24; and a distal head assembly 26 extending from the distal end 24 of the flexible sheath 20.

In some embodiments, the grip portion 14 of the handle device 12 can comprise an elongated contoured body 34 that is shaped to be received with the hand (i.e., palm) of a surgeon such that fingers extend about the contoured body and are receiving within the jaw actuator member 16 for pivotal actuation of jaw actuator member 16. The thumb of the surgeon can extend and engage the cutter actuator member 18 for sliding and/or fore-aft pivoting actuation of the cutter actuator member 18.

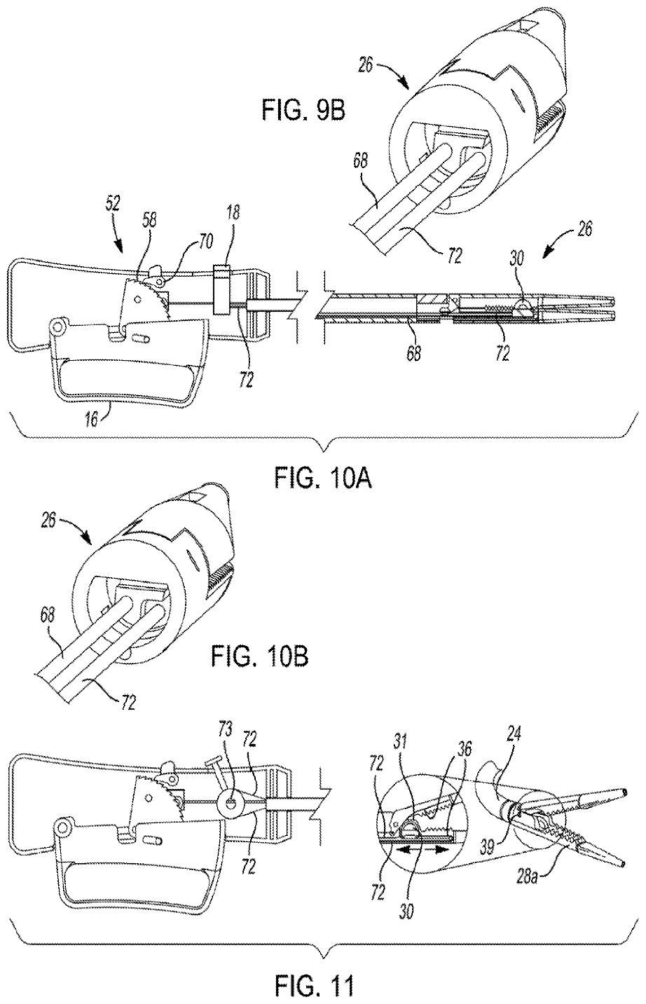

As illustrated in FIG. 4, in some embodiments, the jaw actuator member 16 of the handle device 12 is pivotally coupled to the contoured body 34 at a pivot axis 35. In some embodiments, fingers can be received within an enclosed lever 38 to permit pivotal actuation of jaw actuator member 16 in opposing directions. The jaw actuator member 16 is operably coupled, such as via drive members, cables, wires, or the like, to at least one of the pair of jaw members 28a, 28b to urge the at least one of the pair of jaw members 28a, 28b between the opened (i.e., unclamped) and closed (i.e., clamped) positions. In some embodiments, retraction of the jaw actuator member 16 results in movement of the at least one of the pair of jaw members 28a, 28b into the closed position to retain a clamping force on the septum during a cutting operation. In some embodiments, as illustrated in FIGS. 9A-10B, this closed position configured to retain a clamping force on the septum during the cutting operation can be achieved via a jaw actuation position lock mechanism 52. As illustrated, in some embodiments, jaw actuation position lock mechanism 52 can comprise a ratchet and pawl mechanism 54 coupled to the jaw actuator member 16. This may help aid the user in grasping and holding onto the septum/dissection flap 116. This mechanism can optionally be de-activated as well so the user can release the dissection flap 116 that is held within the jaw members 28a, 28b.

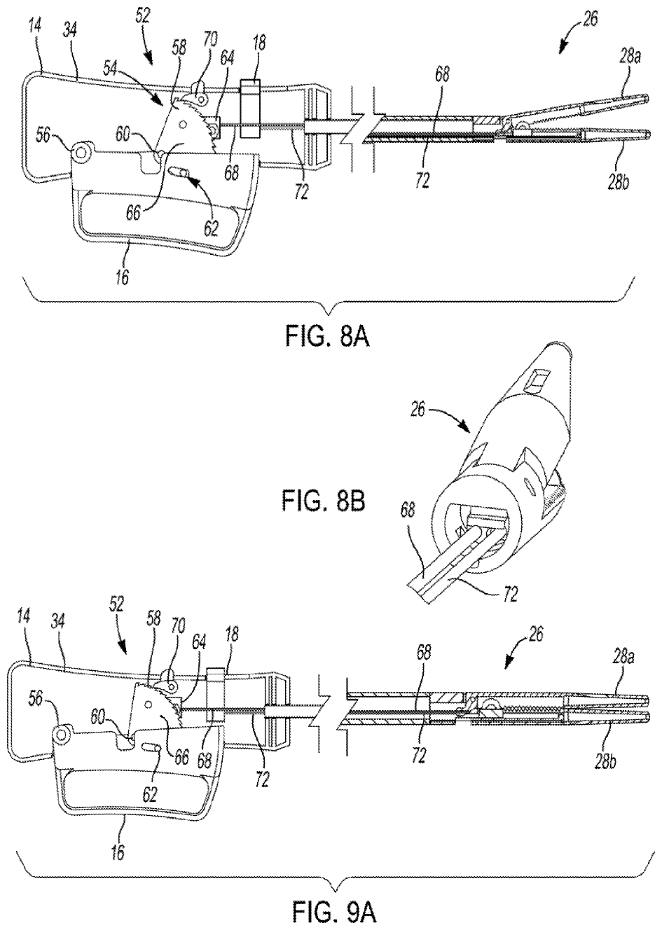

In some embodiments, as illustrated in FIGS. 8A-12B, ratchet and pawl mechanism 54 is operably coupled to the jaw actuator member 16 such that jaw actuator member 16 is pivotally coupled about an axis 56 to rotate in response to actuation by a user. Gripping and compressing of jaw actuator member 16 causes jaw actuator member 16 to rotate (e.g., in a counterclockwise direction of FIGS. 8A, 9A, 10A, 11, and 12B). Ratchet and pawl mechanism 54 can comprise a ratchet member 58 pivotally coupled about axis 60. A cam pin and slot assembly 62 formed between jaw actuator member 16 and ratch member 58 provides camming engagement and rotation of ratch member 58 about axis 60 in response to rotation of jaw actuator member 16 (e.g., in a counterclockwise direction). Ratchet member 58 is operably coupled to a jaw actuation cable mount 64. Jaw actuation cable mount 64 is linearly slidable in response to rotation of ratchet 58 via connection 66. Jaw actuation cable mount 64 is coupled to at least one of the pair of jaw members 28a, 28b via a jaw actuation cable 68 to urge the at least one of the pair of jaw members 28a, 28b between the opened (i.e., unclamped) and closed (i.e., clamped) positions in response to actuation of jaw actuator member 16.

Ratchet and pawl mechanism 54 can further comprise a pawl 70 for selectively lockingly engaging ratchet 58 to retain ratchet 58 in a direction resulting in at least one of the pair of jaw members 28a, 28b in the closed (i.e., clamped) position without the user/surgeon having to maintain gripping force on the jaw actuator member 16. Pawl 70 can be released via thumb feature or other unlocking feature to release the ratchet 58 and permit opening of the jaw members 28a, 28b.

The thumb of the surgeon can extend and engage the cutter actuator member 18 for fore-aft movement, including linear, sliding, pivoting, rotating, actuation of the cutter actuator member 18. In some embodiments, as illustrated in FIGS. 8A, 9A, 10A, and 12B, cutter actuator member 18 can be slidably disposed in gripping portion 14. Cutter actuator member 18 can be operably coupled to a cutter actuation cable 72 that is in turn operably coupled to cutting member 30. It should be understood that cutter actuation cable 72 can comprise a single cable (e.g., FIGS. 8A-10B) or two cables (e.g., FIG. 11). In some embodiments, cutter actuation cable 72 comprises a metal, such as but not limited to stainless steel or nitinol, being either a monofilament or braided configuration. It should also be understood that the two-cable configuration can include a single cable that is routed in two directions to provide tensile pulling force in opposing directions. In this configuration, cutter actuator can comprise fore-aft actuation. As illustrated in FIGS. 8A-10B, in some embodiments, the cutter actuator member 18 of the handle device 12 is slidably coupled to the contoured body 34 on a side opposite of the jaw actuator member 16 to permit fore-aft linear actuation of cutter actuator member 18. As illustrated in FIGS. 4 and 11, in some embodiments, the cutter actuator member 18 of the handle device 12 is pivotally coupled to the contoured body 34 on a side opposite of the jaw actuator member 16 for rotation about axis 73 to permit fore-aft pivoting actuation of cutter actuator member 18. As illustrated in FIGS. 8A-11, the cutter actuator member 18 is operably coupled, such as via drive members, cables, one or more wires, or the like, to cutting member 30 to urge the cutting member 30 along a slot or guide channel 36 formed in distal head assembly 26 (such as, but not limited to, formed in jaw member 28a, jaw member 28b, or both). In some embodiments, cutting member 30 can be a cutting blade having a cutting edge on one or more portions of the cutting member 30. In some embodiments, cutting member 30 can comprise a cutting edge on a single side to permit cutting in only one actuation direction. In some embodiments, cutting member 30 can comprise cutting edges on opposing sides to permit cutting in opposing distal and proximal cutting directions. In some embodiments, cutting member 30 can comprise a cutting edge along an arcuate edge to permit cutting in a plurality of directions. In some embodiments, cutting member 30 can comprise a laser or other form of energy delivery device (i.e., bipolar cutter).

During operation, as illustrated in FIGS. 8A-8B, initially the jaw actuator member 16 is in the open position, allowing the jaw members 28a, 28b to open. The cutter actuator member 18 is in the proximal position, this moves the cutting carriage/cutting member 30 to the proximal location where it is not exposed in a cutting configuration. This helps to minimize the chances of unintentional cutting during navigation which could cause harm to the patient. Note that in these examples the guidewires through the lumen introducer elements are not shown for ease of clarity of showing the mechanisms.

As illustrated in FIGS. 9A-9B, the jaw actuator member 16 is in the closed position, which exerts a tensile pulling force the jaw actuation cable 68. This closed the jaw members 28a, 28b, allowing them to grab and hold the septum, isolating its movement. The cutter actuator member 18 is still in the proximal position, holding the cutting carriage in the proximal location where it is not exposed in a cutting configuration. The septal tissue abuts the tissue stopping element/protective wall 31. The surgeon may now confirm that they are in the proper location and that it is safe to perform the septal cut.

A tissue stopping element/protective wall 31 on the distal end acts as a hard stop for the septal tissue. While the cutting element may slide past the tissue stopping element via, for example, an integrated slot, the septal tissue may not. This ensures that the cutting element can always cut the distal most segment of the septal area, allowing the device to advance proximally along the aortic septum after cutting takes place so that subsequent cutting may occur as needed along the length of the septum.

With the handle still in the closed position and the septum held securely in place, the cutter actuator member 18 can now be moved to the distal position to cause the cutting member 30 to be advanced. This shifts the tension on the cutting carriage actuation cable(s) 72 causing the cutting carriage and cutting member 30 to advance towards the distal position, cutting through that portion of the septal tissue.

After the septum is cut, the cutting carriage can be returned to the non-exposed configuration. The jaw members 28a, 28b can be relaxed and the device can be advanced further through the septum where the septal cutting process may be repeated as many times as necessary for the patient.

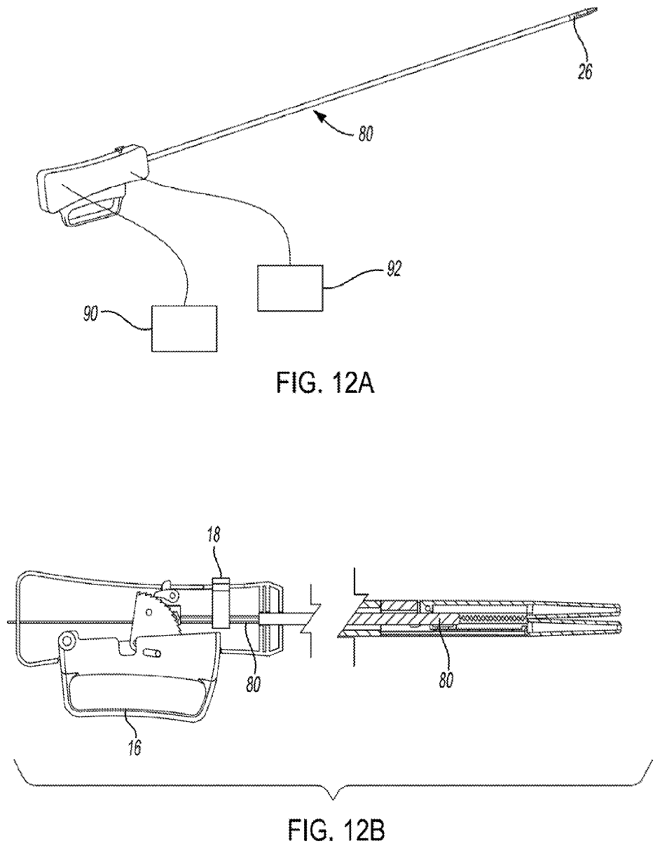

In some embodiments, as illustrated in FIGS. 12A-12B, the cutting member 30 may be replaced with a laser fiber 80 (generically, cutting member 30 and laser fiber 80 are referred to as a cutting mechanism). The laser fiber 80 can comprise a laser source 90 and may be oriented such that the distal tip of the catheter may move back and forth along the cutting carriage and can contact the grasped septal tissue. This laser fiber 80 may be, for example, a 2 mm laser catheter similar to the Turbo Elite Laser Atherectomy Catheter OTW, Philips Inc. To induce septal cutting, the laser may be activated at an appropriate energy level. This may be, for example, approximately 50 mJ/mm² at a rate of 60 pulses per second. The laser fiber 80 may then be translated along the isolated septum via the cutting carriage while active to complete the cutting of that septal section. Further, a vacuum source 92 may be connected to pull vacuum through the flexible sheath. This may help minimize the chances of any coagulated blood or tissue escaping from the grasping mechanism and entering the patient's bloodstream. In some embodiments, the laser fiber 80 may be translated directly using cutter actuator member 18 rather than via the cutting carriage. In some embodiments, the laser fiber 80 may be translated directly by hand by pushing on the laser fiber passing through the back of the device (FIG. 12B)

In some embodiments, cutting member 30 can be received within a parked portion of distal head assembly 26 to prevent cutting of tissue until cutting member 30 has been deployed from the parked portion (i.e., to prevent cutting during delivery or positioning of distal head assembly 26 to the target aortic dissection flap). In this parked position, as illustrated in FIG. 4 cutting member 30 can be stowed or otherwise captured within or by a protective element, wall, or shroud 31 to prevent or inhibit unintended contact of cutting member 30 with tissue. In some embodiments, protective wall 31 (or other separate feature) can serve as a “tissue stop” to provide an indication and/or prevent further advancement of distal head assembly 26 upon reaching uncut septum. In some embodiments, protective wall 31 can facilitate a clean cut of the leading edge of septal tissue. That is, without protective wall 31, uncut tissue may end up proximal to the most proximal position of the cutting member after the cutting member 30 has been activated. If this occurred, the user may have difficulty advancing the distal head assembly 26 along the septum as septal cutting is performed. In some embodiments, cutting member 30 can be parked at a proximal location (see FIG. 4). In some embodiments, cutting member 30 can be parked at a distal location (see FIG. 5, wherein cutting member 30 has been deployed from the distal location). It should be understood that in some embodiments multiple locations can be used to park cutting member 30. Actuation of the cutter actuator member 18 results in movement of the cutting member 30 along the guide channel 36 to exert a cutting force on the septum.

In some embodiments, distal head assembly 26 can comprise the pair of pivotally coupled jaw members 28a, 28b positionable between an opened or unclamping position (see FIGS. 4 and 6) and a closed or clamping position (see FIGS. 5 and 6). In some embodiments, a first 28a of the pair of jaw members can be fixedly coupled to the flexible sheath 20, such that it remains coaxially aligned with the distal end 24 of flexible sheath 20. In some embodiments, flexible sheath 20 is a 16 Fr sheath to 24 Fr sheath. In this way, a second 28b of the pair of jaw members can be pivotally coupled to the first jaw member 28a at a pivot axis 39. Each of the pair of jaw members 28a, 28b can comprise retention features 40 configured to grip and/or retain the dissection flap 116 therein in response to applied clamping force in the closed position. In some embodiments, the retention features 40 can comprise a serrated edge, teeth, or other increased friction feature configured to non-destructively grasp the dissection flap 116. In some embodiments, one or both of the pair of jaw members 28a, 28b can comprise a flexible tip portion 42 to facilitate navigation of the distal head assembly 26 along artery walls to the landing zone as illustrated in FIGS. 4, 6, and 7. In some embodiments, flexible tip portion 42 can be tapered in shape to a distal tip. This tapered shape can further serve to dilate the septal opening during insertion.

In some embodiments, the distal head assembly 26 comprises guidewires 32a, 32b extending within the true lumen 110 and the false lumen 112 to guide the distal head assembly 26 to the target area 114. Particularly, as illustrated in FIGS. 4-7, in some embodiments, each of the pair of jaw members 28a, 28b can comprise a guidewire slot 44 formed in longitudinal direction along the middle of the jaw member. The guidewire slot 44 is sized and shaped to receive one of the guidewires 32a, 32b. In this way, guidewires 32a, 32b extending within the true lumen 110 and the false lumen 112 provide a guide path along which the distal head assembly 26 is directed to the target area 114. More particularly, in some embodiments, the guidewires 32a, 32b can comprise a single guidewire that is inserted from an iliofemoral sheath and delivered to the proximal pre-existing fenestration of the aortic dissection flap using a steerable sheath via the true lumen (or false lumen). A snare catheter is delivered from the same femoral sheath to the false lumen (or true lumen). The guidewire is captured from the false lumen side and brought back to the same femoral sheath. Once this guidewire loop system is established, the distal head assembly 26 is ready to be delivered to the target aortic flap to be cut. Each end of the looped guidewire is inserted into each guidewire slot 44 of the jaw members 28a, 28b. The jaw members 28a, 28b can be rotated as needed.

Once the distal head assembly 26 is delivered to the target area 114, the jaw actuator member 16 is activated and the target flap to be divided or cut is captured by the jaw members 28a, 28b. Next, the cutter actuator member 18 is actuated and the already captured dissection flap 116 is cut by driving the cutting member 30 in response to movement of the cutter actuator member 18 by the surgeon. The cutter actuator member 18 is further actuated to capture the cutting member 30 in the parked position (within or by protective wall 31) and the distal head assembly 26 is further advanced more proximally. The jaw actuator member 16 and the cutter actuator member 18 are again actuated and the flap is further cut.

In some embodiments, this process is performed under fluoroscopic guidance; however, this can be objectively visualized either using IVUS or angioscope to assure the safety of the intra-aortic septal cutting, if necessary. Once the dissection flap is cut sufficiently to create a landing zone for the stent-graft, commercially available endograft is placed and the treatment is completed.

The distal head assembly 26 can be inserted via a pushing motion of the handle device 12. The pair of jaw members 28a, 28b can remain in the opened position. The guidewires 32a, 32b, each extending through one of the pair of jaw members 28a, 28b, serve to guide the distal head assembly 26 to the target area (i.e., the dissection flap extending between the true lumen 110 and the false lumen 112). Following the procedure, the endovascular aortic septotomy device 10 is then removed and the region of the aorta that had the dissection can now equalize pressure in the true and false lumen and provide a more suitable location for a stent-graft.

### Procedural Details

The procedural use of the endovascular aortic septotomy device 10 will now be described in connection with an exemplary treatment procedure.

### Bilateral Femoral Access

A patient is first brought to a hybrid operating room (operating room with C-arm and fluoroscopy) and placed under general anesthesia. The patient is prepped and draped in the standard fashion. Bilateral common femoral arteries are accessed under ultrasound and fluoroscopy guidance using 5 Fr microcatheters. Heparin is administered to achieve Activated clotting time (ACT) 250-300.

Percutaneous vessel access closure device (most commonly Abbott Perclose devices x 2) are placed on the ipsilateral access over 0.035 inch×150 cm J-wire. A 18-24 Fr iliofemoral sheath (depends on the main aortic stent graft size) in the ipsilateral access and 8 Fr sheath (exclusively for intravascular ultrasound (IVUS) usage) in the contralateral access through J-wire.

### Aortic Septotomy

IVUS is inserted from the 8 Fr sheath through J-wire. Initial IVUS examination of the entire aorta is performed. Initial IVUS examination includes confirmation of true vs false lumen access from each femoral access, locations of pre-existing septal fenestrations (which can be utilized as the start point of the aortic septotomy), aorta size measurement at intended landing zones during systole.

From the main sheath, two stiff guidewires (0.035 inch×150 cm) are inserted and one placed in the true lumen and one in the false lumen under fluoroscopy and IVUS guidance. Cross access can be typically obtained from one of the pre-existing fenestrations within the aorta segment between the celiac artery and iliac bifurcation (such as fenestrations from original left renal artery, inferior mesenteric artery, celiac artery, intercostal & lumbar arteries, etc.) with glide catheter or cobra catheter support. In cases where no pre-existing fenestrations are present, an aortic septal fenestration is to be created using needle fenestration technique, laser fenestration technique, or any endovascular technique at the discretion of the operating surgeon. Creating a brand-new fenestration at the straight aorta segment between the celiac and iliac bifurcation level is technically feasible with common endovascular techniques.

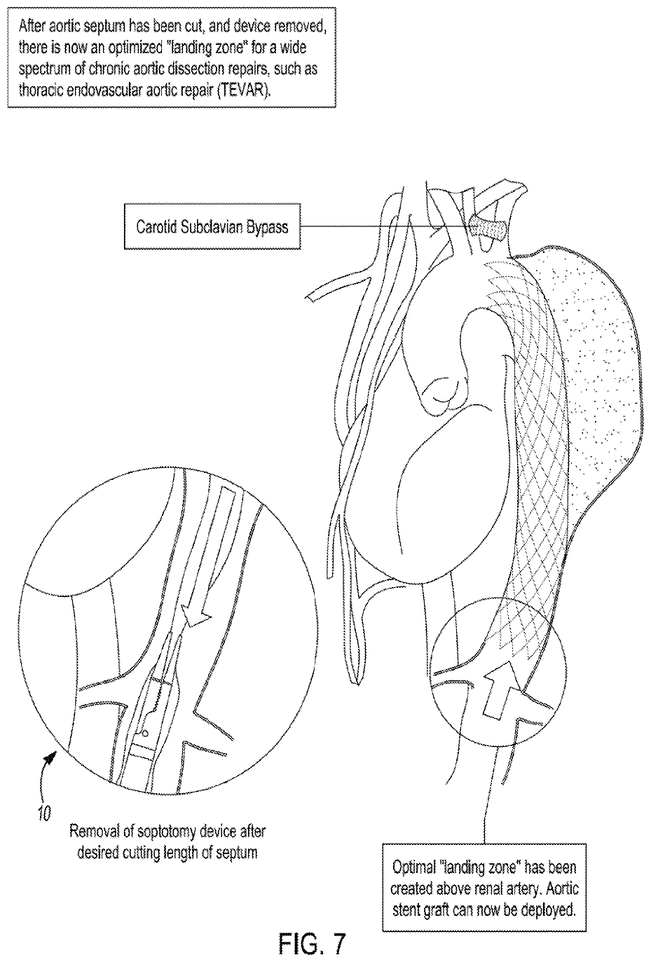

As illustrated in FIGS. 6 and 7, once each stiff glide/guidewire is in place in the true and false lumen, each wire end outside the body is inserted into each guidewire port of the endovascular aortic septotomy device 10. Once each wire is loaded within the guidewire slot 44, the distal head assembly 26 of the endovascular aortic septotomy device 10 is inserted into the ipsilateral main sheath.

The distal head assembly 26 is delivered through these guidewires to the target aortic septum under fluoroscopy guidance. The jaw of the distal head assembly 26 is tapered shape and serves as a stand-alone dilator so that it can go through a small fenestration. In case one jaw of the device gets caught by the aortic septum, the pre-existing septal fenestration may be pre-dilated using standard COOK hydrophilic dilators or 12-18 mm Percutaneous Transluminal Angioplasty (PTA) balloon. The still guidewire may be replaced with Rosen or Amplatz super stiff wire as needed. The device is reinserted afterwards.

The distal head assembly 26 is advanced to the target septum and the jaw actuator member 16 of the endovascular aortic septotomy device 10 is squeezed by the surgeon's hand. The target aortic septum is captured by the pair of jaw members 28a, 28b of the endovascular aortic septotomy device 10. Ensuring the proper capturing the target aortic septum, it should be confirmed by surgeon's hands (gently pulling and feeling resistance), IVUS (confirming the septum captured by the two graspers) and fluoroscopy.

The cutting member 30 is released and the septum is cut by actuating the cutter actuator member 18 by the surgeon's thumb. The pair of jaw members 28a, 28b can then be opened-the cutting member 30 and/or cutter actuator member 18 can be biased to the “parked” configuration via a spring mechanism that pulls the cutting member 30 to the parked position when no force is applied to the cutter actuator member 18. The distal head assembly 26 is proximally advanced through the already placed guidewire in the true and false lumen. Additional septum is cut in the same fashion repeatedly up through the intended landing zone. A 5-10 cm length of cut along the septum is typically adequate to achieve a suitable landing zone for a stent-graft. The distal head assembly 26 is withdrawn and removed from the patient.

Completeness of the septal cutting can be confirmed by IVUS. Once it is confirmed, the false lumen guidewire is withdrawn.

### Main Aortic Stent Graft Placement

The true lumen guidewire is advanced via glide catheter to the ascending aorta. IVUS catheter is withdrawn from the 8 Fr sheath. A 0.035 inch×100 cm omni catheter is inserted from the contralateral 8 Fr sheath using J wire. The omni catheter (diagnostic pigtail catheter) is positioned in the ascending aorta.

A 0.035 inch 260 cm (or 300 cm) extra stiff straight Lunderquist wire is inserted via the glide catheter to the ascending aorta. The glide catheter is removed. Main aortic stent graft is inserted from the ipsilateral access and advanced to the aortic arch via the Lunderquist wire.

C-arm is positioned at the pre-determined deployment angle. Diagnostic aortography is performed from the omni catheter at a rate of 20 cc/second totaling of 30 cc contrast. The locations of the head-vessels are marked on the screen.

Aortic stent graft is deployed. The delivery system is withdrawn from the access. Additional stent grafts are deployed as needed.

Completion aortography was performed through the omni catheter in the same fashion.

The femoral sheath is removed over a J wire and perclose close devices on the ipsilateral access are tied. Another perclose device is placed as needed for the contralateral access. Protamine is given to reverse heparin.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. An aortic septotomy device for cutting aortic tissue between a true lumen and a false lumen, the aortic septotomy device comprising:
a handle device having a jaw actuator member;
a distal head assembly having a pair of jaw members movable between an opened position and a clamping position and a cutting mechanism movable within a channel; and
a sheath member extending from the handle device to the distal head assembly;
wherein at least one of the pair of jaw members is movable in response to actuation of the jaw actuator member to selectively grasp aortic tissue, and the cutting mechanism is movable within the channel to cut at least a portion of the aortic tissue while the pair of jaw members grasps the aortic tissue.

2. The aortic septotomy device according to claim 1 wherein the handle device further comprises a cutter actuator member operable to move the cutting mechanism within the channel in response to actuation of the cutter actuator member.

3. The aortic septotomy device according to claim 2 wherein the cutter actuator member is configured to be coupled to a body portion of the handle device for fore-aft actuation to drive the cutting mechanism within the channel.

4. The aortic septotomy device according to claim 1 further comprising:
a first guidewire configured to extend within the true lumen;
a second guidewire configured to extend within the false lumen,
a first guidewire slot formed in a first of the pair of jaw members and a second guidewire slot formed in a second of the pair of jaw members, the first guidewire extending through the first guidewire slot to guide movement of the first jaw member within the true lumen and the second guidewire extending through the second guidewire slot to guide movement of the second jaw member within the false lumen.

5. The aortic septotomy device according to claim 1 wherein the handle device is configured to be grasped by a surgeon and the jaw actuator member is configured to be pivotally coupled to a body portion of the handle device for pivotal movement in opposing directions.

6. The aortic septotomy device according to claim 1 wherein the cutting mechanism is configured to cut in a single direction.

7. The aortic septotomy device according to claim 1 wherein the cutting mechanism is configured to cut in at least two directions.

8. The aortic septotomy device according to claim 1 wherein at least one of the pair of jaw members is generally coaxial with a distal end of the sheath member.

9. The aortic septotomy device according to claim 1 further comprising:
a flexible tip portion extending from at least one of the pair of jaw members.

10. The aortic septotomy device according to claim 8 wherein the flexible tip is tapered.

11. The aortic septotomy device according to claim 1 wherein the distal head assembly comprises a tissue stop member selectively contacting aortic tissue and configured to align the cutting mechanism relative to uncut aortic tissue.
